(19) United States
(12) Patent Application Publication
(10) Pub. No.: US 2026/0105859 A1
(43) Pub. Date: Apr. 16, 2026

(54) ASSESSMENT OF CLINICAL PROCEDURE QUALITY AND SKILL SYSTEMS AND METHODS

(71) Applicant: Northwestern Memorial Healthcare, Chicago, IL (US)

(72) Inventors: Mozziyar ETEMADI, Chicago, IL (US); James Alex HELLER, Chicago, IL (US); Evandros KAKLAMANOS, Chicago, IL (US); Rajesh KESWANI, Wilmette, IL (US); Matthew KLUG, Chicago, IL (US); Kristjana KRISTINSDOTTIR, Chicago, IL (US); Matthew WITTBRODT, Chicago, IL (US)

(21) Appl. No.: 18/915,920

(22) Filed: Oct. 15, 2024

(51) Int. Cl. G09B 19/00; G06Q 10/0639; G16H 50/20

(52) U.S. Cl. CPC G09B 19/00; G06Q 10/06398; G16H 50/20

(57) ABSTRACT

Systems and methods include one or more machine learning systems to identify skills within clinical data, compare the identified skills to one or more metrics, and provide a determination of provider quality based, at least in part, on the one or more metrics. An interactive environment may be provided in which practitioners can provide clinical data for evaluation and then receive additional feedback on training data to determine areas of improvement. The skills evaluated by the system may be correlated to positive clinical outcomes, thereby providing systems and methods to improve overall practitioner quality.

### BACKGROUND

### 1. Field of Disclosure

Embodiments of the present disclosure relate to systems and methods to evaluate metrics associated with imaging procedures, including but not limited to endoscopy. Specifically, one or more embodiments are directed toward automated methods to assess metrics associated with quality or skill in performing diagnostic evaluations and/or treatments.

### 2. Description of Related Art

Medical providers may perform various procedures when evaluating and/or treating an interior of a patient's body. For example, diagnostic evaluations may include one or more procedures were images are acquired of an interior of the patient's body, such as by inserting a camera or using other imaging devices, and/or samples may be taken from an interior, such as by removing issues. Additionally, treatments may also include removal of tissue or other actions. When obtaining images for diagnostic and/or treatment procedures, images may be helpful and acquiring such images may take various forms, including but not limited to, endoscopy in which an endoscope (e.g., a probe) is inserted into a hollow organ or cavity of a body. Endoscopy may include the insertion of the endoscope directly into the organ and the endoscope may include one or more imaging devices, such as a camera, along with other tools that may be particularized for a given procedure. Endoscopy is generally performed by a practitioner and quality may vary based on a variety of factors, such as experience level, time, and the like. Quality can be improved with additional training and feedback, but doing so is often a manual process that requires a practitioner with sufficient skill level to fully review and then provide feedback on the procedure, which may not be feasible or may be subject to biases or otherwise subjective evaluations.

### SUMMARY

Applicant recognized the problems noted above herein and conceived and developed embodiments of systems and methods, according to the present disclosure, for analysis of endoscopy, which may be an automated process using one or more machine learning systems.

In an embodiment, a system includes a trained machine learning system to identify, from input clinical data, one or more features of interest associated with a skill corresponding to a clinical procedure. The system also includes a skill evaluation system to determine a metric associated with the skill, the metric being an indicator of a quality of performance for the skill. The system further includes a training system configured to provide feedback, responsive to a request, regarding at least a portion of the skill to identify one or more actions associated with the skill to be modified by a practitioner performing the skill.

In another embodiment, a method includes receiving input clinical data for a clinical procedure. The method also includes identifying, from the input clinical data, one or more skills associated with a clinical outcome. The method further includes determining, based on one or more metrics, a quality level for the one or more skills. The method also includes providing a score for a practitioner associated with the clinical procedure based, at least in part, on the quality level.

In an embodiment, a processor includes one or more circuits to receive input clinical data for a clinical procedure. The one or more circuits may also identify, from the input clinical data, one or more skills associated with a clinical outcome. The one or more circuits may further determine, based on one or more metrics, a quality level for the one or more skills. The one or more circuits may provide a score for a practitioner associated with the clinical procedure based, at least in part, on the quality level.

### BRIEF DESCRIPTION OF DRAWINGS

The present technology will be better understood on reading the following detailed description of non-limiting embodiments thereof, and on examining the accompanying drawings, in which:

FIG. 1 illustrates an environment for analysis of clinical data, in accordance with embodiments of the present disclosure;

FIG. 2A illustrates an environment for parameter identification and training, in accordance with embodiments of the present disclosure;

FIG. 2B illustrates an example environment for skill and quality identification and training, in accordance with embodiments of the present disclosure;

FIG. 3 illustrates an example interface for providing feedback from a clinical procedure, in accordance with embodiments of the present disclosure;

FIG. 4 is a flow chart of a process for rating a practitioner performing a clinical procedure, in accordance with embodiments of the present disclosure;

FIG. 5 is a flow chart of a process for providing feedback and training for a clinical procedure, in accordance with embodiments of the present disclosure; and

FIG. 6 is an example configuration for a computing device, in accordance with embodiments of the present disclosure.

### DETAILED DESCRIPTION

The foregoing aspects, features, and advantages of the present disclosure will be further appreciated when considered with reference to the following description of embodiments and accompanying drawings. In describing the embodiments of the disclosure illustrated in the appended drawings, specific terminology will be used for the sake of clarity. However, the disclosure is not intended to be limited to the specific terms used, and it is to be understood that each specific term includes equivalents that operate in a similar manner to accomplish a similar purpose. Additionally, like reference numerals may be used for like components, but such use should not be interpreted as limiting the disclosure.

When introducing elements of various embodiments of the present disclosure, the articles “a”, “an”, “the”, and “said” are intended to mean that there are one or more of the elements. The terms “comprising”, “including”, and “having” are intended to be inclusive and mean that there may be additional elements other than the listed elements. Any examples of operating parameters and/or environmental conditions are not exclusive of other parameters/conditions of the disclosed embodiments. Additionally, it should be understood that references to “one embodiment”, “an embodiment”, “certain embodiments”, or “other embodiments” of the present disclosure are not intended to be interpreted as excluding the existence of additional embodiments that also incorporate the recited features. Furthermore, reference to terms such as “above”, “below”, “upper”, “lower”, “side”, “front”, “back”, or other terms regarding orientation or direction are made with reference to the illustrated embodiments and are not intended to be limiting or exclude other orientations or directions. Like numbers may be used to refer to like elements throughout, but it should be appreciated that using like numbers is for convenience and clarity and not intended to limit embodiments of the present disclosure. Moreover, references to “substantially” or “approximately” or “about” may refer to differences within ranges of +/−10 percent.

Embodiments of the present disclosure are directed towards systems and methods to receive, analyze, rate, and provide feedback for one or more clinical procedures associated with a diagnostic and/or treatment operation for a patient. In at least one embodiment, clinical procedures may refer to one or more skills used in diagnostic and/or treatment. For example, skills may include viewing an image, providing a diagnostic evaluation based on the image, obtaining the image, obtaining a video feed, performing one or more tasks associated with the diagnostic and/or treatment procedure, and/or combinations thereof. In at least one embodiment, systems and methods may be directed toward one or more quality metrics associated with different portions of or steps in a diagnostic and/or imaging procedure. By way of non-limiting example, quality metrics associated with viewing an image may be directed toward evaluating a quality of whether or not a practitioner has properly (e.g., within some threshold level of skill) evaluated by an image, such as determining whether a practitioner has correctly identified abnormalities within an image, whether a practitioner has improperly identified abnormalities within an image (e.g., false positives), whether a practitioner has manipulated a tool in order to obtain sufficient image data, whether a practitioner has performed a task within threshold level of skill, and/or the like. In one example, different diagnostic and/or treatment operations may be performed along with, and/or may include, one or more imaging processes, including by not limited to endoscopy. In at least one embodiment, one or more images, such as a sequence of images (e.g., frames) provided as part of a video file, may be evaluated by one or more machine learning systems to identify different metrics that may be associated with quality of a service provided by a practitioner associated one or more portions of a procedure, such as an endoscopy. For example, evaluation of the frames may be associated with an evaluation of a quality of skill associated with viewing and/or analyzing the frames, obtaining the frames, or performing a task that is then viewed within the frames, among other options and combinations thereof. The system may be used to identify different features, correlate these features to the different metrics based on one or more rules, and then provide an evaluation and/or output associated with these metrics to provide feedback and guidance for one or more actions that may be used to improve or otherwise increase a quality evaluation associated with the procedure. In certain embodiments, a review of a procedure may be automated or substantially automated such that different portions or features are identified for further review or analysis, such as a sequence of frames characterized as being associated with a certain procedure or action performed during the procedure. In this manner, a quality metric may be assessed for a provider, or for a portion of the procedure, to provide guidelines and/or areas of improvement. Accordingly, procedure quality can be improved for a variety of different practitioners with reduced human input.

Various embodiments may be directed toward diagnostic and/or treatment procedures, such as endoscopy. One such non-limiting example may be gastrointestinal endoscopy (e.g., colonoscopy and upper endoscopy), but it should be appreciated that various other diagnostic and/or treatment techniques may benefit from the systems and methods described herein. As non-limiting examples, diagnostic and/or treatment techniques may include a variety of applications that may join or otherwise incorporate imaging along with one or more actions by a practitioner, such as surgical operations where an imaging device, such as a camera, is used during treatment (e.g., surgery, diagnostic procedures, etc.) and may then be evaluated in real or near-real time, or after the fact, to assess one or more quality metrics for the associated tasks performed and captured by the imaging device. Furthermore, various procedures in which imaging devices are deployed may also benefit from various embodiments of the present disclosure, such as surgical procedures that use an imaging device to guide one or more portions of the procedure. By way of non-limiting example, endoscopy review may be performed on the gastrointestinal tract (e.g., esophagus, stomach, duodenum, small intestine, large intestine/colon, magnification endoscopy, bile duct, rectum, anus, etc.), the respiratory tract (e.g., nose, upper respiratory tract, lower respiratory tract, etc.), the ear, the urinary tract, the female reproductive system (e.g., cervix, uterus, fallopian tubes, etc.), the male reproductive system, and/or cavities accessible via one or more incisions that are otherwise closed (e.g., abdominal, pelvic cavity, joints, organs of the chest, etc.). Furthermore, endoscopy may also be used during or prior to one or more procedures, including but not limited to, pregnancy (e.g., amnion, fetus, etc.), plastic surgery, Panendoscopy (a combination of laryngoscopy, esophagoscopy, and bronchoscopy), orthopedic surgery (e.g., hand surgery, endoscopic carpal tunnel release, knee surgery, anterior cruciate ligament reconstruction, epidural space, bursae, etc.), endodontic surgery (e.g., maxillary sinus, apicoectomy), endoscopy endonasal surgery, and endoscopic spinal surgery, among others. An endoscopy may include using a tool or device that includes one or more imaging devices, such as a camera, and one or more additional tools or apparatuses, depending on the procedure. For example, the endoscope may include a cutting tool or a tool to perform different actions or operations, such as tissue biopsies, banding, snaring, inserting, cauterizing, removal, dilation, and/or the like. Furthermore, the endoscope may be used in combination with other tools to guide operations that include additional tools. For example, an endoscope may be used to provide a view of an interior portion of a vein or artery for insertion of a stent or other device, among various other procedures. Accordingly, while systems and methods may be described toward specific procedures associated with endoscopy for gastrointestinal or esophageal procedures, it should be appreciated that various embodiments may be used in a variety of situations in which imaging systems may provide information relevant for evaluating one or more metrics associated with a quality of a service provided.

Systems and methods of the present disclosure recognize that quality (e.g., a measure associated with a procedure that may be linked or otherwise correlated to a clinical outcome) varies between providers. For example, a highly skilled provider may be more likely to identify a cancerous growth than a less skilled provider. Similarly, a highly skilled provider may be more likely to successfully perform a surgical operation in a high-risk area than a less skilled provider. However, these skills can be improved by delivering feedback on technical skills (e.g., how well a provider is performing one or more tasks) and also measuring clinical outcomes. Such assessments of technical skill and outcomes is challenging in practice at least due to the high level of manual review to clearly identify and link different skills to metrics associated with a likelihood of improved clinical outcomes. Systems and methods address these problems, among others, by using one or more tools, that may include trained machine learning (ML) systems and/or artificial intelligence (AI), to automate review and evaluation of quality and/or quality of one or more procedures or operations associated with different diagnostic and/or treatment procedures, such as those that use endoscopes or other imaging devices. As a result, technical skill may be quickly analyzed to identify areas for improvement and provide feedback to practitioners to improve their skills. Additionally, skills and clinical outcome may be tracked over time to identify correlations between certain actions and clinical outcomes to improve identification of various metrics. Furthermore, in at least one embodiment, interactive training may be provided for users. Moreover, in at least one embodiment, real or near-real time (e.g., without significant delay) analysis may be incorporated into the systems and methods to monitor a procedure as it occurs to provide feedback or improvements during the procedure, thereby increasing a likelihood of a high quality score and associated improved clinical outcomes.

Various embodiments incorporate one or more trained ML systems to measure key metrics and features based, at least in part, on expert-based guidelines identifying different skills or actions associated with high quality and/or successful clinical outcomes. In at least one embodiment, a particular clinical outcome may be correlated to certain actions or tasks performed during a procedure. For example, a practitioner that spends more time in a certain bodily region may have an increased likelihood of identifying a problem within that region, such as a polyp, and therefore, one metric may be associated with how long a practitioner spends within a region as a threshold level of quality correlated to a positive clinical outcome. In another example, the practitioner's use of a tool, such as rotations per minute to identify a 360 degree view of a particular organ, or a rate at which the tool is advanced, among other options, may also be correlated to or associated with one or more metrics. By way of further example, the practitioner's use of different secondary tools, such as to band or cauterize certain identified irregularities, may also be associated with a quality metric. In this manner, a set of rules may be established and then deployed when analyzing different portions of a procedure, such as by identifying a series of frames (e.g., within a video segment) associated with one or more actions. In at least one embodiment, one or more classifiers may be incorporated to identify features of interest (e.g., polyps, growths, anomalies, use of tools, etc.) and/or to identify and mark certain actions or skills performed by practitioners (e.g., cleaning, performing a biopsy, removing an object, etc.). These features may be identified within a given frame and then certain actions or associations may be tracked and used to correlate these features and/or associated actions to different metrics. For example, a duration of time to remove a growth may be tracked, among other options. In this manner, systems and methods may provide for automated review and labeling or tracking of actions, which may then further be reviewed by different systems, for example in association with an output generated in which certain actions are categorized and presented for rapid viewing and to provide feedback to practitioners.

In at least one embodiment, systems and methods may be incorporated into an interactive application (e.g., a web-based application) that may be used to integrate features (e.g., a user interface, access to the ML systems, etc.) into a dashboard or singular location where the quality of one or more procedures associated with endoscopy or other imaging, surgical, or diagnostic operations can be evaluated. For example, systems and methods may be executed to review, classify, and categorize portions of the procedure (e.g., endoscopy), such as different portions of a video, and then a reviewer and/or a physician can scroll through the video to identify the features identified by trained ML systems. Furthermore, multiple procedures for a single practitioner may be assessed and aggregated to determine overall clinical quality. Additionally, during the course of a procedure, different actions may be scored, weighted, and then used to provide an overall quality score for the practitioner for a given procedure. Over time, quality may be tracked to identify where the practitioner is improving or weakening, which may inform future training or identify areas where new equipment or technologies may be helpful in increasing quality scores.

Various embodiments of the present disclosure may integrate the features of the systems and methods into a dashboard or platform for providing services to providers, such as those associated with a health system or the like. Different levels of instruction and/or operability may be provided, such as for training, aggregate measurement, review, and/or the like. For example, a trainer and/or trainee can utilize systems and methods, for example within an interactive dashboard or interface, to review a procedure and identify areas that require remediation or improvements. For example, if biopsies are being performed during an upper endoscopy procedure, the system can highlight where biopsy technique might be optimal (e.g., satisfies a threshold associated with one or more metrics) and/or a trainer can review the video with a trainee and explain why the biopsy technique was not optimal (e.g., below a threshold associated with one or more metrics). Furthermore, embodiments may also be used to aggregate measurements of technical skill and quality metrics for one or more physicians. For example, a small hospital might want to measure the skill of a physician performing colonoscopies. Using recorded videos, embodiments could produce an aggregate assessment of that physician. Additionally, systems and methods may also be deployed to review prior endoscopic procedures to see if concerning clinical features may be identified to inform or otherwise discuss during patient follow-up. For example, the tool might determine when the appropriate diagnostic evaluation was not performed for a particular diagnosis (e.g., celiac disease). Accordingly, systems and methods may be used to provide a variety of different types of services to providers, including training, review, analysis, and follow on evaluation.

Systems and methods overcome problems with existing techniques, including at least the difficulties in accurately assessing quality of service providers and/or identifying correlations between certain actions and clinical outcomes, among others. For example, prior systems may evaluate measurements of endoscopy outcomes with manual abstraction (e.g., chart review) in which a human reviewer looks through charts to determine outcomes. While electronic health record integration has automated some of this process, these records are limited with respect to quality metrics that are recorded, which may miss or otherwise disregard important features that can only be obtained from review of the imaging results. Furthermore, measurement of technical skills (e.g., how a physician is looking at the stomach during an upper endoscopy, etc.) cannot readily be automated, and as a result, is generally performed by a clinical reviewer watching one or more endoscopy videos and giving a qualitative assessment of skill. This is both laborious and prone to reviewer biases. Thus, it is only done rarely in the research setting despite its value in improving clinical care. Embodiments of the present disclosure overcome these problems, and others, to provide systems and methods for use with training, diagnosis, and analysis of skill as it correlates to clinical outcomes.

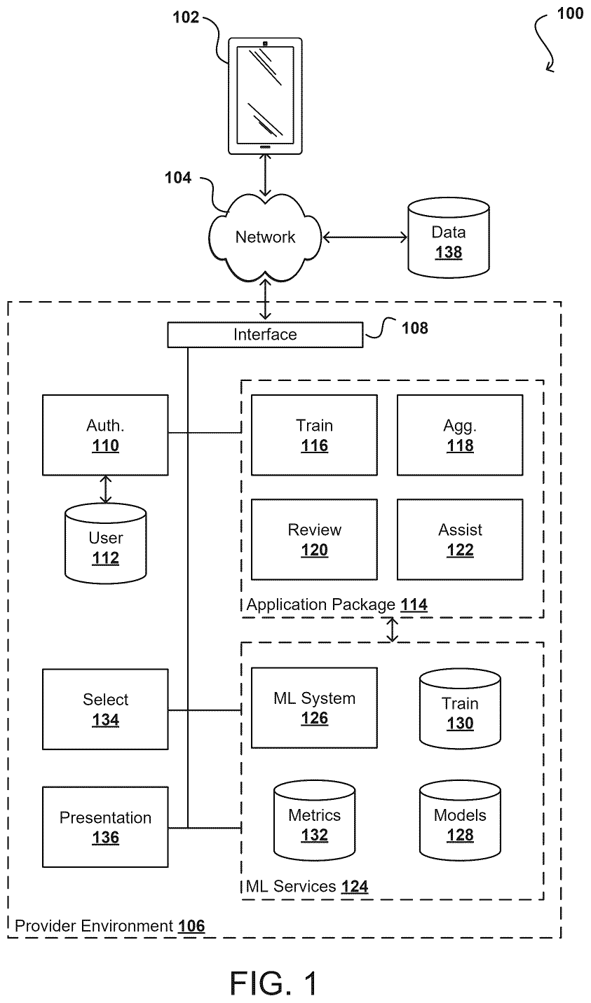

FIG. 1 illustrates an example system 100 that may be used with embodiments of the present disclosure. In this example, a computing device 102 (e.g., user device, compute device, client device, etc.) can submit a request at least one network 104 to be received by a provider environment 106. The provider environment 106 may be an online platform provided by a service provider and/or for an affiliate, for example the environment 106 may be hosted or otherwise provided via one or more cloud resource providers on behalf of a service provider. The client computing device 102 may be representative and/or act as a proxy for one or more users that may be submitting requests. For example, a user may navigate to one or more dashboards, web applications, landing pages, or access points using the device to submit a request, among other options. Additionally, in at least one embodiment, the client computing device 102 may act as a proxy to execute stored instructions to make and receive requests. For example, the client computing device 102 may send a request responsive to receiving one or more inputs and/or the like. As another example, a request may be transmitted as part of an automated or semi-automated workflow, which may or may not receive user interaction. Accordingly, the client computing device 102 may be used with direct input from one or more users, from stored software instructions, from executions of various workflows, or combinations thereof.

In at least some embodiments, the request can include a request to execute one or more workflows associated with analysis and/or processing of electronic health records (EHR), including evaluation of imaging data, among other options. In many cases, this will include a request to access data (e.g., stored data, streaming data, etc.) and then to process the data using one or more workflows associated with the environment 106. In at least one embodiment, a selected workflow may be based, at least in part, on information provided by the computing device 102, such as a command, or based on data received by the environment 106. The network(s) 104 can include any appropriate network, such as the Internet, a local area network (LAN), a cellular network, an Ethernet, or other such wired and/or wireless network. The provider environment 106 can include any appropriate resources for accessing data or information, such as EHR, as may include various servers, data stores, and other such components known or used for accessing data and/or processing data from across a network (or from the “cloud”). Moreover, the client computing device 102 can be any appropriate computing or processing device, as may include a desktop or notebook computer, smartphone, tablet, wearable computer (e.g., smart watch, glasses, contacts, headset, etc.), server, or other such system or device.

An interface layer 108, when receiving a request or call, can determine the type of call or request and cause information to be forwarded to the appropriate component or sub-system. For example, the interface 108 may be associated with one or more landing pages, as an example, to guide a user toward a workflow or action. In at least one embodiment, the interface layer 108 may include other functionality and implementations, such as load balancing and the like.

Various embodiments of the present disclosure are directed toward processing and/or evaluation of EHR, among other features, and as a result certain data protection operations may be deployed. In at least one embodiment, an authentication service 110 may be associated with the provider environment 106 to verify credentials provided by the client device 102, for example against a user database 112, to verify and permit access to the environment 106. Furthermore, in at least one embodiment, verification may also determine a level of accessibility within the environment 106, which may be on an application-basis, a user-basis, or some combination thereof. For example, a first user may have access to the environment, but only have a limited set of applications that are accessible, while a second user may have access to more applications, and a third user may be entirely barred from the environment. In this manner, access may be controlled and information related to EHR may be protected.

Systems and methods may include a web-based or application-based portal that permits receipt, analysis, and evaluation of information, such as EHR, that may include multi-modal information including text data, video data, image data, audio data, and/or combinations thereof. In at least one embodiment, an application package 114 may be executable within the environment 106 to provide functionality to the client device 102 according to one or more embodiments. In this example, the application package 114 may be associated with different sets of executable software instructions, which may share one or more underlying resources, to perform one or more tasks. The applications illustrated in FIG. 1 are provided by way of non-limiting example, and include at least a training package 116, an aggregated measurement package 118, a review package 120, and an assistance package 112. These packages 114 may be provided as a software suite where the computing device 102 may submit a request to access one or more packages 114 to analyze or otherwise interact with information provided by the computing device 102, such as EHR.

As noted herein, various embodiments may be directed toward EHR, such as image data obtained from one or more diagnostic and/or treatment tools, including by way of non-limiting example, an endoscope. While embodiments may be described with respect to endoscopes and endoscopy, it should be appreciated systems and methods may be extended to various other applications and tools. In this example, information may be provided to the environment 106 that includes data, which may be image data (e.g., video, still images, diagnostic notes), audio data (e.g., recorded audio, audio extracted from a video, auditory notes generated before or during the procedure, etc.), textual data (e.g., notes inputted before, after, or during the procedures), and/or combinations thereof associated with one or more diagnostic and/or treatment procedures. As discussed herein, the diagnostic and/or treatment procedures may optionally combine imaging devices and/or tools that may be used to perform certain actions on the patient undergoing the treatment, such as tools to interact with the patient. Furthermore, the one or more tools may be used to manipulate or otherwise adjust the imaging device, such as to advance or rotate a camera, among other options. The user may wish to provide the information to the application package 114 in order to analyze or otherwise provide feedback on different aspects within the data, such as providing feedback on techniques used to perform the procedure, aggregating and determining a quality metric for a provider, reviewing information within the data to assist in diagnostics and/or follow up, and/or to provide rear or near-real time assistance, among various other options. Various embodiments may incorporate one or more ML systems to receive and process the data to identify different features or interest. These features of interest may then be correlated with a diagnosis and/or with one or more skills or tasks performed by a practitioner during the procedure that produced the data. These features may be identified, such as by using one or more classifiers associated with different feature identification algorithms and methods, among other options, and then aggregated to provide feedback for the practitioner based, at least in part, on one or more metrics. The metrics may provide thresholds to evaluate different skills or tasks against and then may be used to guide or otherwise correct different skills or tasks performed by practitioners. In at least one embodiment, the metrics may be tied or otherwise correlated to clinical results, which may then be used to improve performance and skill of different practitioners.

In at least one embodiment, systems and methods of the present disclosure deploy one or more ML services 124, which may include execution of different software instructions based, at least in part, on a request received from the user device. In this example, the ML services 124 may include different ML systems 126 that may be used to execute one or more models 128 to accomplish a given task. These models 128 may be trained on different training data 130, which may be labeled or unlabeled, and also may be guided by one or more metrics 132, which may be used as parameters when executing different training operations. As discussed herein, training data may be augmented or otherwise influenced by one or more human reviewers, but it should be appreciated that raw training data may be used with one or more self-supervised learning processes.

During operation, a selection service 134 may be used to provide information to the ML services 124 to choose a particular model of the models 128 for use with the different application packages 114. For example, one or more models for training 116 may be different than models used when performing aggregation 118. Accordingly, systems and methods may modify or otherwise adjust which models are selected, for example, based on the type of data received, the application the user selects, and/or the like. Furthermore, systems may also include a presentation service 136 to provide information to the user in a visually pleasing and/or interactable format. By way of non-limiting example, for training purposes, the user may be presented with frames of a video that have been tagged and coded with various operations that may be associated with areas where the practitioner performed at or above a threshold level of competency and others where the practitioner performed below a threshold level of competency. Furthermore, the presentation service 136 may be used to visualize data, such as providing information where practitioners excel and where they fall behind, thereby providing information to develop a targeted training platform to raise overall competency. Furthermore, the presentation service 136 may also be used to generate various reports and the like.

In operation, a user associated with the device 102 may be a provider of one or more diagnostic and/or treatment services, such as endoscopy as one example. The user may wish to access an available platform in order to receive training and feedback related to a procedure. The user may submit a request to the environment 106, which may include providing data 138 acquired during the procedure, such as image data, diagnostic notes, prior information for the patient, and/or the like. Based on the information, the user may use the selection system 134 to select the training package 116, which may then cause one or more different models 128 to be executed by the ML system 126. In at least one embodiment, the data 138 may be processed for use by the ML system 126, such as by segmenting the images, color correction, or other techniques. Thereafter, if the data includes video data as one example, different frames may be evaluated by the models 128 to identify features of interest, which may be associated with diagnostic information, technique information, skill data, or other parameters associated with the different models 128.

In at least one embodiment, the data 138 may correspond to endoscopy data that includes video data acquired during a colonoscopy. The ML system 126 may use the model 128 to identify features of interest and/or skills used during the procedure, such as identification (e.g., using one or more classifiers) of polyps, identification of the use of different tools (e.g., snares, cauterizers, etc.), identification of certain portions of the colon (e.g., a region based on features associated with that region, a region based on a time stamp for a given image, etc.), and/or different skills or actions performed during the procedure (e.g., cleaning, rotating, speed of movement, etc.). These different identified actions may then be time stamped or coded within the sequence of images (e.g., frames) forming the video data for the procedure and may be correlated to different factors or metrics. For example, an insertion time may be monitored based on data collected from the images. As another example, other parameters may also be identified, such as withdrawal time, cecum frequency, polyps found, polyps missed, polyps removed, total time, and/or the like. These parameters may then be evaluated against one or more metrics to determine a skill level for the user. The metrics may be developed based on clinical outcomes, such as identifying a reasonable or average amount of total time, identifying clinically significant withdrawal times, and/or the like. Thereafter, parameters or statistics for the user may be flagged and an interactive hub may be provided where the user can identify different actions or features for the procedure, receive feedback regarding positive and negative actions, and then be provided with tips or suggestions for improvement. For example, a user can be shown an instance where they performed a certain technique “correctly” (e.g., according to the one or more metrics) to compare to other less desirable applications of the technique. For example, with a training module, an interactive application may provide a side-by-side comparison of the “correct” technique and the “incorrect” technique in order to specifically map errors or insufficiencies. As one example, the application may illustrate how a tool was improperly navigated, such as by identifying movement that drove the tool into a tissue region instead of through an opening. As another example, the application may identify a missed polyp or illustrate a region where movement of the imaging device was too rapid. In this manner, the user may improve their skills, which may be tracked over a number of procedures, such as using the aggregation package 118, to track their skill level and identify areas of high skill or low skill, which may suggest additional training is needed to increase an overall level of skill.

Various embodiments may also use the packages 114 to analyze information for particular providers and/or for systems as a whole. For example, continuing with the example of EHR, an affiliate provider for a hospital system may have four providers that each perform certain endoscopy procedures. Their overall data may be aggregated to determine a level of the affiliate provider, and moreover, each individual provider within the affiliate provider may also be evaluated to identify high quality providers. The hospital system may encourage improvements or maintenance above a certain level to maintain affiliate status, and as a result, may improve patient care and overall clinical results.

In at least one embodiment, the review package 120 may be used to evaluate prior procedures to identify potential errors or missed information, which may be relevant for use with follow up procedures. For example, if a provider missed a polyp or performed part of the procedure in a manner that was below certain metrics, follow up procedures may be recommended or more tracking may be used for patient care. Additionally, various embodiments may also enable execution of the packages in real or near-real time, for example the assistance package 122 may receive streaming video for an ongoing imaging procedure, such as an endoscopy, and may provide real or near-real time feedback, such as identification of abnormalities, instructions (e.g., slow down, rotate the tool, etc.), and/or the like. In this manner, improved patient care may be provided by using the various systems of the environment 106.

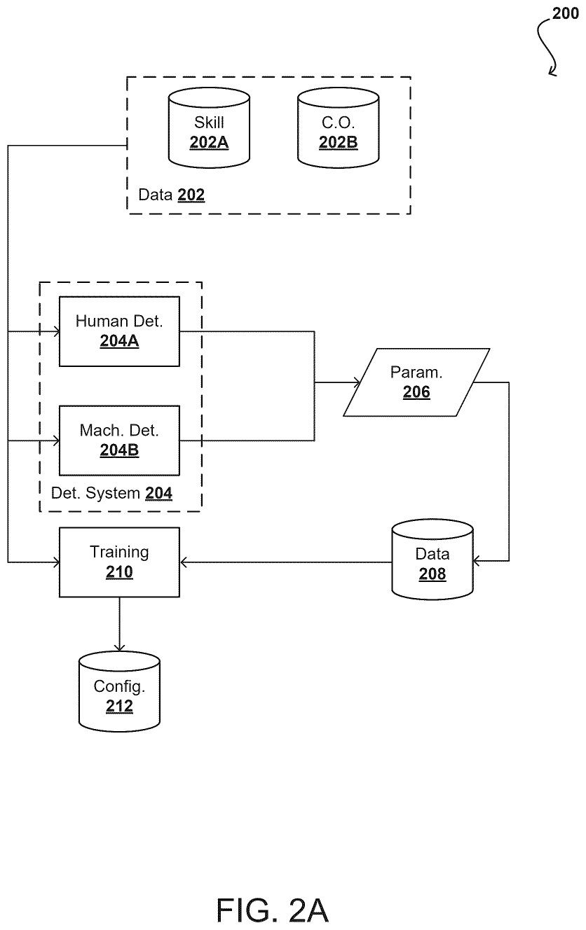

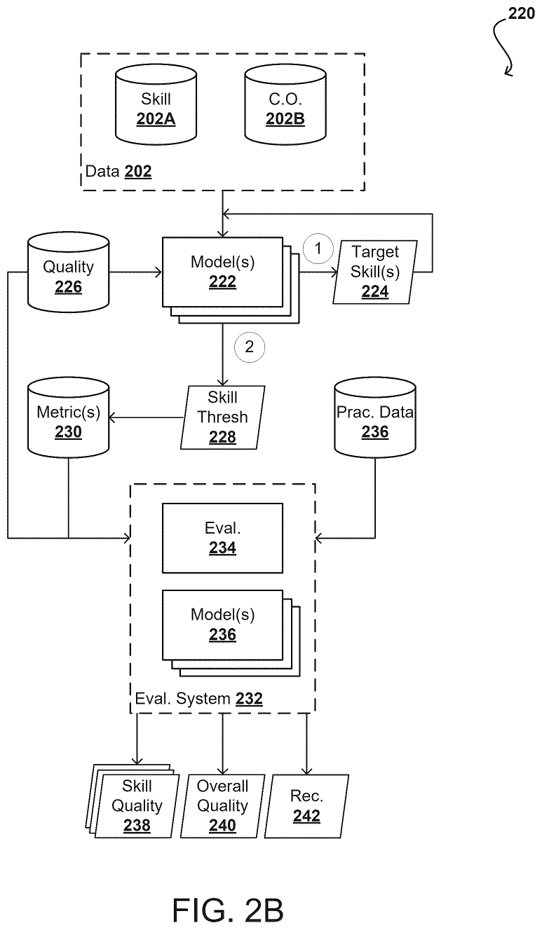

FIG. 2A illustrates an example environment 200 in which parameters related to skills correlated to positive clinical outcomes may be determined and used to train one or more machine learning systems. In this example, clinical data 202 may include various databases of aggregated information, such as EHR, which includes information such as skill data 202A and clinical outcome data 202B. For example, skill data may correspond to actions taken by a practitioner during a procedure, such as a speed at which certain actions are performed, use of certain tools, a duration of time spent in a target region, and/or the like. Skills may be aggregated over time for different procedures and may be particularized for different procedures, which may lead to development of different models for different procedures. For example, the skills performed during a colonoscopy may be different from the skills performing during heart surgery, and as a result, correlations may be adjusted based on desired clinical outcomes, expert assessment of skills, and/or the like. Clinical outcome data 202B may associate different actions taken by a practitioner with certain outcomes. For example, a highly skilled practitioner may have better clinical outcomes (e.g., earlier detection of cancer, reduced complications after surgery, etc.) than a less skilled practitioner. By aggregating data from different practitioners over time, and correlating different skills to clinical outcomes, systems and methods of the present disclosure may develop different metrics or parameters that correlate certain skills with desirable outcomes. For example, a metric determination system 204 may be used, which may use humans 204A or ML systems 204B, or combinations thereof, to evaluate skills and their associated clinical outcomes to develop one or more parameters 206 that may be used to identify a particular skill that is determinative or otherwise correlated to a particular clinical outcome.

The identified parameters 206 may then be provided to a parameter database 208, which may include different threshold levels for the parameters 206 (e.g., a duration longer than “X”), which may be used with a training process 210. For example, one or more ML systems may be trained to both identify the particular skills and the associated parameters extracted from the use or application of the skills. Skills may be identified in a number of ways, such as ML classifiers that may identify certain features or objects of interest (e.g., polyps, deployment of tools, etc.) and/or may use state information to determine a first frame in which a region of interest is entered or investigated and then determine when that region of interest is passed in order to identify a time period associated with the investigation. In this manner, a set of configuration parameters 212 (e.g., weights) for an ML system may be developed. Different configuration parameters 212 may be associated with different skills, different procedures, different designed clinical outcomes, and/or the like. Furthermore, a set of parameters may be aggregated into a common system.

FIG. 2B illustrates an example environment 220 that may be used with embodiments of the present disclosure. In this example, the data 202, such as the skills data 202A and/or the clinical outcome data 202B may be evaluated using one or more models 222 to identify correlations between particular skills and/or clinical outcomes. For example, in at least one embodiment, the clinical outcomes may be associated with a transient or temporary result of performing one or more actions associated with a skill (e.g., successfully using a tool, spending a threshold period of time reviewing certain image data, successfully identifying an anomaly, etc.). Additionally, clinical outcomes may also be associated with longer time results, such as a reduced instance of surgical intervention based on certain diagnostic evaluations, an increased likelihood of recovery based on certain intervention techniques, and/or the like. Embodiments of the present disclosure may be used to evaluate the data 202 to determine whether one or more skills are more closely related to or associated with desirable clinical outcomes. The one or more models 222 may include one or more deep neural networks that may receive, as an input, skills and corresponding clinical outcomes and then generate one or more target skills 224, as illustrated by the numeral 1.

The target skills 224 may be associated with certain skills that are found to closely or positively relate to desirable clinical outcomes. For example, successful polyp removal may be considered a positive clinical outcome, and as a result, a skill associated with a tool, such as a snare, for polyp removal may be correlated to a desired clinical outcome and represent a target skill. In at least one embodiment, the one or more models 222, which may be the same or different models, may use quality data 226 to generate one or more skill thresholds 228, as illustrated by the numeral 2. The skill thresholds 228 may as associated with a duration of time, a use of a skill, a detection percentage, and/or combinations thereof. For example, returning to the example of a snare, a duration of time using a snare may not be relevant, but an outcome result of the snare, such as a percentage of polyp removed, may be relevant. As a result, the different skill thresholds may be correlated to particular skills and may not be common or duplicative across skills. The various thresholds for different skills may be considered metrics for evaluation and stored within a metrics datastore 230. The metrics may also be supplemented, or edited, by one or more human reviewers, such as expert practitioners. In this manner, the practitioner may be used to adjust, add, or subtract information associated with the skill thresholds 228 in the datastore 230.

Systems and methods of the present disclosure may be used to generate different assessments or evaluations of practitioners using one or more evaluation systems 232. For example, the evaluation systems 232 may include an evaluation engine 234 that uses one or more models 236 (which may be obtained from one or more datastores and/or may use portions of the models 222). The evaluation engine 234 may receive input practitioner data from a practitioner datastore 238 and then, using the one or more models 236, may evaluate different aspects or portions of the data to generate a variety of outputs. For example, the practitioner data may correspond to video data of a diagnostic and evaluation procedure. The one or more models 236 may be used to identify the operation of execution of certain skills (e.g., diagnostic skills, tool performance, etc.) and evaluate the skills against different metrics to identify one or more scores or outputs. The outputs, in this example, including a skill quality score 238, an overall quality score 240, and a recommendation 242. The skill quality score 238 may be an evaluation of the skill within the practitioner data and the quality and/or metrics. There may also be multiple scores for a certain skill. For example, if a skill was associated with use of a tool, there may be multiple metrics such as duration of use, quality of use, outcome of use, and/or the like. The overall quality score 240 may aggregate one or more portions, or an entirety, of the practitioner data. For example, the overall quality score 240 may represent collection of each use of a particular tool and an aggregate score. In at least one embodiment, the recommendation 242 may provide an evaluation of the different scores 238, 240 against one or more thresholds and provide a recommendation or other evaluation, such as a recommendation to continue training in a certain area and/or an evaluation as a highly skilled practitioner. In this manner, information may be used over a number of procedures to identify target skills, establish metrics for the skills, and then analyze and evaluation different operations in view of the skill sand metrics to score practitioners and/or their quality with respect to different skills and/or clinical outcomes.

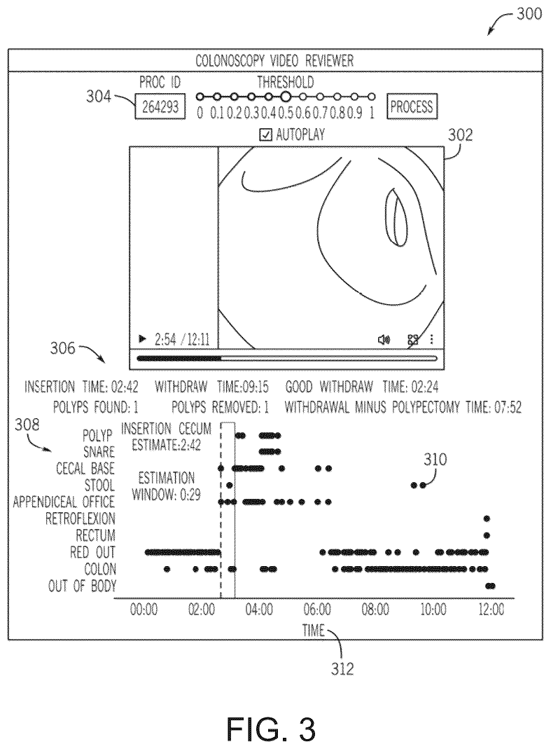

FIG. 3 illustrates an example interface 300 which may be generated using systems and methods of the present disclosure. In this example, the interface 300 includes a visual indicator 302 of a frame of a video sequence, for example from an endoscopy procedure, such as a colonoscopy. Further shown on the interface 300 includes an identifier 304 for a given practitioner. In operation, the sequence of frames may be processed and one or more features 306 may be identified that may correlate to specific positive clinical outcomes. This example shows certain features 306 such as “Insertion Time” and “Withdrawal Time,” among various others. It should be appreciated that these features may be considered highly relevant, and therefore, may be singularly noted. Additionally, it should be appreciated that these features may be associated with parameters that cannot be marked or otherwise identified in individual frames, or in which such marking is difficult, and therefore are associated with overall parameters or features of the procedure. Furthermore, there may be a mix of the two, such as including both “Polyps Found” and also “Cecum Frequency” in the list. In at least one embodiment, the certain feature 306 may be provided as a drop menu and/or may be presented separate from other features discussed herein. For example, certain features may be considered salient and most dispositive with particular desirable outcomes, and as a result, the interface may be designed to highlight these features. However, it should be appreciated that the features may also be tunable so that a practitioner and/or an institution may select those which are most important and/or focus on particular features, such as during a certain stage of training.

Additionally, a set of features 308 is shown with associated markers 310 along a time 312 of the procedure. Particular markers 310 may be acquired during processing via the machine learning systems, for example by identifying a polyp. Additionally, clinical notes may also be used for such correlations. In at least one embodiment, a multi-modality system may be used to identify features within the image, such as a polyp, with notes (e.g., textual, audio, etc.) provided by the practitioner before, during, and/or after the procedure. For example, during treatment, the practitioner may announce and verbally describe a polyp and the steps taken to remove the polyp. This information may then be correlated to visual analysis of a frame of the treatment to provide an accurate notation for the event corresponding to the marker. Additionally, different actions associated with those findings may be shown as overlapping. For example, in this case, the polyp identification overlaps with the use of the snare, indicating that the polyp was removed or otherwise treated using the snare. After use of the snare, the polyp is no longer identified, which provides an indication of successful removal. Further examples of such features may also be shown in FIG. 3, such identification of the cecal base and the appendiceal orifice substantially overlapping due to their locations relative to one another. Additionally, the markers 310 may show multiple views of certain portions due to withdrawal time, movement of the endoscope, and other features. For example, if an imaging device is being moved through a section of the colon, the device may be stopped or linger on a region while the practitioner evaluates a region or ensures identification of one or more objects of interest, such as an abnormality that may be desirable to view from multiple angles.

The illustrated features 308 may be interactive such that the image 302 illustrated is tied to a selection of the one or more markers 310. For example, the user may select a marker 310 and a frame of that action or a sequence of frames in the form of a video may be playable within the interface 300. In at least one embodiment, one or more warnings or flags may also accompany different markers 310, indicating that the skill used was below a threshold. For example, if polyp removal was unsuccessful, a warning may be provided so that the practitioner can review the attempted removal and, in certain embodiments, also view a video of successful removal so that they can learn by seeing other improved techniques. In this manner, the user may be trained not only by providing feedback on their own procedures but also by viewing successful or higher skilled practitioners.

Various embodiments of the present disclosure may also provide an output report for a practitioner, which may rate or otherwise provide an indication of their apparent skill level and identifying areas for improvement. For example, the user may be provided with a list of skills and their rating compared to a metric. This may be used to target additional training. Furthermore, such systems may be used by hospital systems to identify which affiliates to recommend patients to and/or which affiliates to continue their relationships.

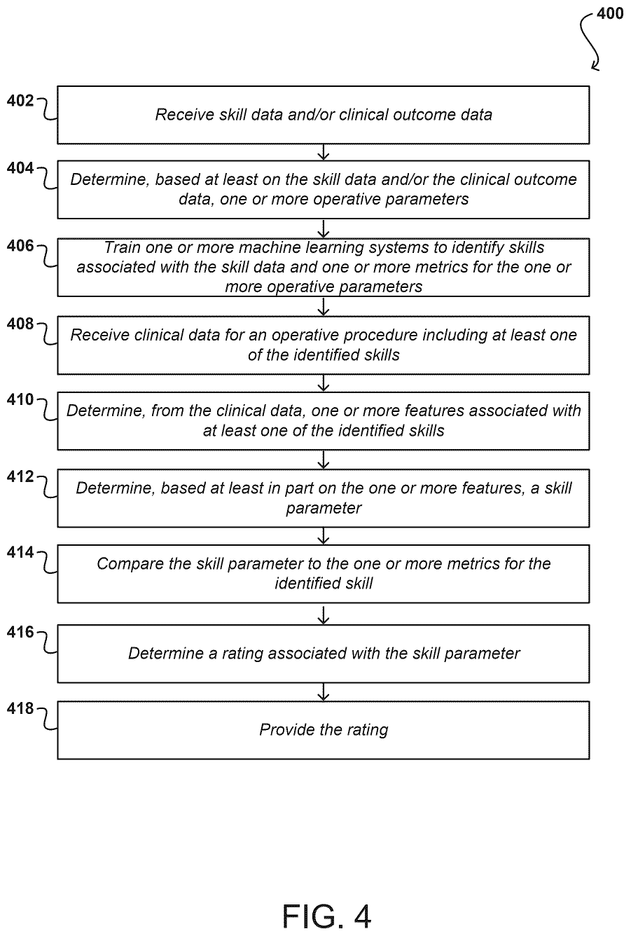

FIG. 4 illustrates an example process for rating a practitioner based on an identified skill. It should be appreciated that steps for the method may be performed in any order, or in parallel, unless otherwise specifically stated. Moreover, the method may include more or fewer steps. In this example, skill data and/or clinical outcome data are received 402. For example, data may be aggregated over a number of practitioners for one or more skills performed during certain procedures and their associated clinical outcomes. However, as discussed herein, certain embodiments may also be associated with training systems that may omit clinical outcome data, such as training for how to operate a tool. In at least one embodiment, the skill data and/or the clinical outcome data may be used to determine one or more operative parameters 404. The operative parameters may correlate skills to certain outcomes, such as a duration performing a task to a positive outcome or lack of performing a task to a negative outcome, among various other options. Moreover, the operative parameters may be associated with groups of skills and/or with thresholds. Additionally, in at least one embodiment, operative parameters may be associated with a successful or unsuccessful completion of a task tied to a skill. For example, if a training operation were developed to teach a practitioner to use a snare to remove a polyp, the clinical outcome may not be used and, instead, the operative parameters may be associated with the use of the snare and/or the outcome of snare use (e.g., removal).

One or more ML systems may be trained on the skill data, the one or more operative parameters, and/or one or more metrics for the operative parameters 406. For example, a skill may be associated with removal of a polyp during a colonoscopy. The operative parameters may include use of a particular tool. And the one or more metrics may be a time using the tool or an outcome associated with the use, such as a percentage of removal, among other options. Additionally, the machine learning systems may include various different models that learn different aspects that can be grouped to perform a task. Returning to the polyp example, a classifier may be trained to identify the polyp, additionally, one or more additional classifiers, or the same classifier, may be trained to identify particular tools. Furthermore, another system may be trained to monitor a time or use. Accordingly, various embodiments may be used to train and/or use the models in a variety of different diagnostic and/or treatment scenarios. Additionally, it should be appreciated that, in certain embodiments, one or more parameters and/or metrics may be determined by one or more ML systems. That is, one or more ML systems may determine a correlation between actions and clinical outcomes to identify a particular metric or parameter, which may further be used to train one or more additional ML systems for identification and evaluation of such actions. As discussed herein, the clinical outcomes may be overall patient outcomes (e.g., patient recovery rates or complications due to performing or not performing an action) or may be outcomes from use of a particular tool or skill. For example, a cauterizing operation may have a clinical outcome associated with a success or failure of the operation with respect to bleeding and/or the like.

Clinical data may be received for an operative procedure that includes at least one of the identified skills from the skill data 408. For example, an imaging procedure may include video data, auto data, clinical notes, and/or the like. The clinical data may be processed to determine one or more features associated with the identified skills 410. For example, feature recognition may be used to identify various skills or items of interest during different frames of the clinical data in embodiments where the clinical data includes images or video data. A skill parameter may then be determined 412. The skill parameter may be associated with use a tool, a duration of time using a tool, a duration of time performing an identified action, and/or the like. The skill parameter may then be compared to one or more metrics 414 and, based on that comparison, a rating may be determined for the particular skill associated with the parameter 416. For example, a practitioner may be rated as highly skilled where they identify and quickly remove a polyp or where they spend more than a threshold time evaluating a particular region of the body. Conversely, a practitioner may be related as less skilled with frequent or more than a threshold number of instances associated with misidentification of an abnormality or if the practitioner fails to perform a common action. The rating may then be provided 418 for further evaluation, such as identification of areas where additional training may be beneficial.

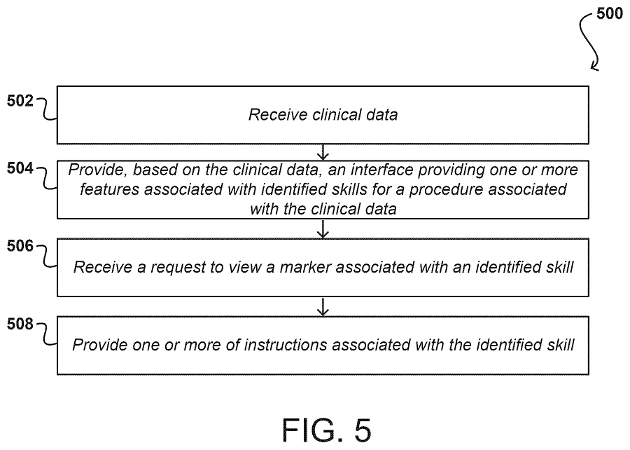

FIG. 5 is a flow chart of a process 500 for providing feedback from a clinical procedure. Clinical data may be received 502 and then processed using one or more ML systems to identify features associated with identified skills for the clinical procedure 504. For example, video data may be received and the machine learning systems may identify different skills associated with the procedure of the video data. A request may be received to request to view a marker associated with an identified skill 506. The marker may provide an indicator where the skill is present and/or may be a warning or identifier where a skill was below a threshold or otherwise did not meet one or more metrics. Responsive to the request, one or more instructions may then be provided 508. For example, video information may be provided illustrating where the skill was performed, an example of a highly skilled use of the skill may be provided, written instructions for the skill may be provided, and/or combinations thereof. In this manner, users may self-train and self-evaluate their procedures to receive feedback and areas for potential improvement.

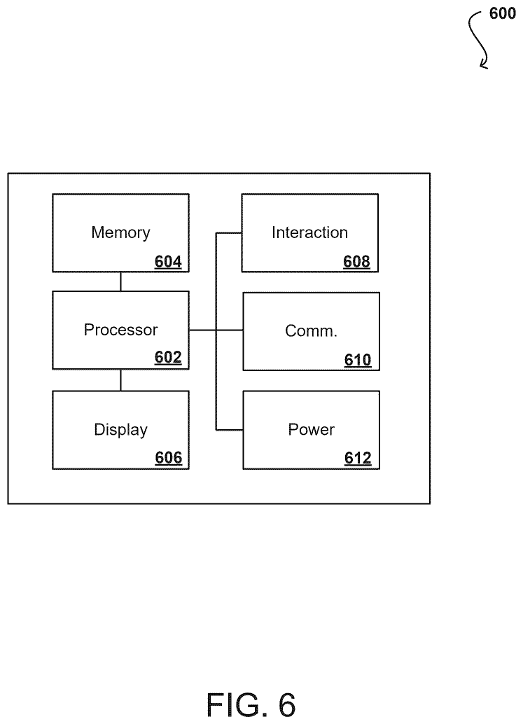

FIG. 6 illustrates a set of general components of an example computing device 600. In this example, the device includes a processor 602 for executing instructions that can be stored in a memory 604. The device can include many types of memory, data storage, or non-transitory computer-readable storage media, such as a first data storage for program instructions for execution by the processor 602, a separate storage for images or data, a removable memory for sharing information with other devices, etc. The device may optionally include a display element 606, such as a touch screen or liquid crystal display (LCD), although devices such as portable media players might convey information via other means, such as through audio speakers, and other devices may not include displays, such as server components executing within data centers, among other options. As discussed, the device in many embodiments will include at least one interaction component 608 able to receive input from a user. This input can include, for example, a push button, touch pad, touch screen, wheel, joystick, keyboard, mouse, keypad, or any other such device or element whereby a user can input a command to the device. In some embodiments, however, such a device might not include any buttons at all, and might be controlled only through a combination of visual and audio commands, such that a user can control the device without having to be in contact with the device. In some embodiments, the computing device 600 of FIG. 6 can include one or more network interface or communication components 610 for communicating over various networks, such as a Wi-Fi, Bluetooth, RF, wired, or wireless communication systems. The device may be configured to communicate with a network, such as the Internet, and may be able to communicate with other such devices. The device will also include one or more power components 612, such as power cords, power ports, batteries, wirelessly powered or rechargeable receivers, and the like.

Storage media and other non-transitory computer readable media for containing code, or portions of code, can include any appropriate media known or used in the art, including storage media and communication media, such as but not limited to volatile and non-volatile, removable and non-removable media implemented in any method or technology for storage of information such as computer readable instructions, data structures, program modules, or other data, including RAM, ROM, EEPROM, flash memory or other memory technology, CD-ROM, digital versatile disk (DVD) or other optical storage, magnetic cassettes, magnetic tape, magnetic disk storage or other magnetic storage devices, or any other medium which can be used to store the desired information and which can be accessed by the a system device. Based on the disclosure and teachings provided herein, a person of ordinary skill in the art will appreciate other ways and/or methods to implement the various embodiments.

Embodiments may also be described in view of the following clauses:
1. A system, comprising:
a trained machine learning system to identify, from input clinical data, one or more features of interest associated with a skill corresponding to a clinical procedure;
a skill evaluation system to determine a metric associated with the skill, the metric being an indicator of a quality of performance for the skill; and
a training system configured to provide feedback, responsive to a request, regarding at least a portion of the skill to identify one or more actions associated with the skill to be modified by a practitioner performing the skill.
2. The system of clause 1, wherein the clinical data includes at least image data of the clinical procedure.
3. The system of clause 1, wherein the clinical procedure is at least one of a diagnostic procedure or a treatment procedure.
4. The system of clause 1, wherein the metric includes a threshold based, at least in part, on a correlation between the quality of performance for the skill and a later observed clinical outcome.
5. The system of clause 1, wherein the training system comprises:
an interface;
a list of the one or more actions; and
a plurality of markers, associated with performance of the one or more actions, selectable by a user.
6. The system of clause 5, wherein the interface, responsive to a selection made by the user, is configured to render at least one of an image or a video sequence corresponding to the one or more actions associated with the selection.
7. The system of clause 1, wherein the skill evaluation system includes one or more trained machine learning systems to determine, based on historical data, at least one of the metric or the quality.
8. The system of clause 1, wherein the training system is configured to provide an instruction associated with at least the portion of the one or more skill to increase the metric.
9. A method, comprising:
receiving input clinical data for a clinical procedure;
identifying, from the input clinical data, one or more skills associated with a clinical outcome;
determining, based on one or more metrics, a quality level for the one or more skills; and
providing a score for a practitioner associated with the clinical procedure based, at least in part, on the quality level.
10. The method of clause 9, further comprising:
identifying one or more features of interest from the input clinical data; and
determining, from the one or more features of interest, the one of more skills.
11. The method of clause 9, further comprising:
receiving a training request, the training request including identification of an action associated with a skill of the one or more skills having a respective quality level below a threshold; and
providing, responsive to the training request, one or more training parameters for the action.
12. The method of clause 11, wherein the one or more training parameters include a video of the one or more skills, an instruction associated with the one or more skills, or a combination thereof.
13. The method of clause 9, wherein the score is an aggregated score based on a plurality of skills.
14. The method of clause 9, further comprising:
receiving historical data for the clinical procedure;
training one or more machine learning systems, using at least the historical data, to identify at least one of the one or more skills; and
training the one or more machine learning systems to generate the quality level and the score.
15. The method of clause 9, wherein the input clinical data includes at least one of image data, video data, audio data, or text data.
16. A processor, comprising:
one or more circuits to:
   receive input clinical data for a clinical procedure;
   identify, from the input clinical data, one or more skills associated with a clinical outcome;
   determine, based on one or more metrics, a quality level for the one or more skills; and
   provide a score for a practitioner associated with the clinical procedure based, at least in part, on the quality level.
17. The processor of clause 16, wherein the one or more circuits are further to:
identify one or more features of interest from the input clinical data; and
determine, from the one or more features of interest, the one of more skills.
18. The processor of clause 16, wherein the one or more circuits are further to:
receive a training request, the training request including identification of an action associated with a skill of the one or more skills having a respective quality level below a threshold; and
provide, responsive to the training request, one or more training parameters for the action.
19. The processor of clause 18, wherein the one or more training parameters include a video of the one or more skills, an instruction associated with the one or more skills, or a combination thereof.
20. The processor of clause 16, wherein the score is an aggregated score based on a plurality of skills.
21. A system, comprising:
a trained machine learning system to identify, from input clinical data, one or more features of interest associated with a skill corresponding to a clinical procedure;
a skill evaluation system to determine a metric associated with the skill, the metric being an indicator of a quality of performance for the skill; and
a training system configured to provide feedback, responsive to a request, regarding at least a portion of the skill to identify one or more actions associated with the skill to be modified by a practitioner performing the skill.
22. The system of clause 21, wherein the clinical data includes at least image data of the clinical procedure.
23. The system of any of clauses 21 or 22, wherein the clinical procedure is at least one of a diagnostic procedure or a treatment procedure.
24. The system of any of clauses 21-23, wherein the metric includes a threshold based, at least in part, on a correlation between the quality of performance for the skill and a later observed clinical outcome.
25. The system of any of clauses 21-24, wherein the training system comprises:
an interface;
a list of the one or more actions; and
a plurality of markers, associated with performance of the one or more actions, selectable by a user.
26. The system of clause 25, wherein the interface, responsive to a selection made by the user, is configured to render at least one of an image or a video sequence corresponding to the one or more actions associated with the selection.
27. The system of any of clauses 21-26, wherein the skill evaluation system includes one or more trained machine learning systems to determine, based on historical data, at least one of the metric or the quality.
28. The system of any of clauses 21-27, wherein the training system is configured to provide an instruction associated with at least the portion of the one or more skill to increase the metric.
29. A method, comprising:
receiving input clinical data for a clinical procedure;
identifying, from the input clinical data, one or more skills associated with a clinical outcome;
determining, based on one or more metrics, a quality level for the one or more skills; and
providing a score for a practitioner associated with the clinical procedure based, at least in part, on the quality level.
30. The method of clause 29, further comprising:
identifying one or more features of interest from the input clinical data; and
determining, from the one or more features of interest, the one of more skills.
31. The method of any of clauses 29 or 30, further comprising:
receiving a training request, the training request including identification of an action associated with a skill of the one or more skills having a respective quality level below a threshold; and
providing, responsive to the training request, one or more training parameters for the action.
32. The method of clause 31, wherein the one or more training parameters include a video of the one or more skills, an instruction associated with the one or more skills, or a combination thereof.
33. The method of any of clauses 29-32, wherein the score is an aggregated score based on a plurality of skills.
34. The method of any of clauses 29-33, further comprising:
receiving historical data for the clinical procedure;
training one or more machine learning systems, using at least the historical data, to identify at least one of the one or more skills; and
training the one or more machine learning systems to generate the quality level and the score.
35. The method of any of clauses 29-34, wherein the input clinical data includes at least one of image data, video data, audio data, or text data.
36. A processor, comprising:
one or more circuits to:
   receive input clinical data for a clinical procedure;
   identify, from the input clinical data, one or more skills associated with a clinical outcome;
   determine, based on one or more metrics, a quality level for the one or more skills; and
   provide a score for a practitioner associated with the clinical procedure based, at least in part, on the quality level.
37. The processor of clause 36, wherein the one or more circuits are further to:
identify one or more features of interest from the input clinical data; and
determine, from the one or more features of interest, the one of more skills.
38. The processor of any of clauses 36 or 37, wherein the one or more circuits are further to:
receive a training request, the training request including identification of an action associated with a skill of the one or more skills having a respective quality level below a threshold; and
provide, responsive to the training request, one or more training parameters for the action.
39. The processor of any of clause 36-38, wherein the one or more training parameters include a video of the one or more skills, an instruction associated with the one or more skills, or a combination thereof.
40. The processor of any of clauses 36-39, wherein the score is an aggregated score based on a plurality of skills.

Although the technology herein has been described with reference to particular embodiments, it is to be understood that these embodiments are merely illustrative of the principles and applications of the present technology. It is therefore to be understood that numerous modifications may be made to the illustrative embodiments and that other arrangements may be devised without departing from the spirit and scope of the present technology as defined by the appended claims.

1. A system, comprising:
a trained machine learning system to identify, from input clinical data including a video having a sequence of frames, one or more features of interest associated with a skill corresponding to a clinical procedure, the skill being identified within a segmented portion of a frame of the sequence of frames and assigned at least one time stamp, wherein the skill is correlated, using one or more trained classifiers and based on one or more identified features within the segmented portion of the frame, to one or more of an identified object in the frame, an identified tool in the frame, or an identified parameter associated with a body part depicted in the frame;
a skill evaluation system to determine a metric associated with the skill, the metric being an indicator of a quality of performance for the skill, and the metric being selected based on at least one of a correlation between the skill and a clinical outcome, a correlation between the skill and a recommendation, or a performance factor associated with performing the skill; and
a training system configured to provide feedback, responsive to a request, regarding at least a portion of the skill to identify one or more actions associated with the skill to be modified by a practitioner performing the skill, the one or more actions directed toward features to increase a skill quality score associated with the metric, wherein the features are based, at least in part, on the skill in the frame.

2. The system of claim 1, wherein the skill is coded based on an individual action of the one or more actions corresponding to the skill.

3. The system of claim 1, wherein the clinical procedure is at least one of a diagnostic procedure or a treatment procedure.

4. The system of claim 1, wherein the metric includes a threshold based, at least in part, on a correlation between the quality of performance for the skill and a later observed clinical outcome.

5. The system of claim 1, wherein the training system comprises:
an interface;
a list of the one or more actions; and
a plurality of markers, associated with performance of the one or more actions, selectable by a user.

6. The system of claim 5, wherein the interface, responsive to a selection made by the user, is configured to render at least one of an image or a video sequence corresponding to the one or more actions associated with the selection.

7. The system of claim 1, wherein the skill evaluation system includes one or more trained machine learning systems to determine, based on historical data, at least one of the metric or the quality.

8. The system of claim 1, wherein the training system is configured to provide an instruction associated with at least the portion of the one or more skill to increase the metric, the instruction including a rendered side-by-side comparison between a first technique associated with the skill and a target technique associated with the skill.

9. A method, comprising:
receiving input clinical data for a clinical procedure, the input clinical data including at least a sequence of frames corresponding to video data collected during the clinical procedure;
identifying, from one or more segmented regions from at least a portion of the sequence of frames using one or more trained classifiers, one or more features of interest and associated time stamps corresponding to one or more actions for performing one or more skills, wherein the one or more skills are correlated to one or more of an identified object in the one or more segmented regions, an identified tool in the one or more segmented regions, an identified parameter associated with a body part depicted in the one or more segmented regions, or a correlation between the associated time stamps and the body part depicted in the one or more segmented regions;
determining, based on one or more metrics and the associated time stamps for the one or more skills, a quality level for the one or more skills, the quality level corresponding to the one or more actions associated with the one or more skills over a period of time identified by a presence of the one or more features of interest across a plurality of time stamps, and the one or more metrics being selected based on at least one of a correlation between the one or more skills and the clinical outcome, a correlation between the one or more skills and a recommendation, or a performance factor associated with performing the one or more skills; and
providing a score for a practitioner associated with the clinical procedure based, at least in part, on the quality level.

10. The method of claim 9, further comprising:
determining, from the one or more features of interest, a starting time for the one of more skills.

11. The method of claim 9, further comprising:
receiving a training request, the training request including identification of an action coded within the one or more segmented regions associated with a skill of the one or more skills having a respective quality level below a threshold; and
providing, responsive to the training request, one or more training parameters for the action.

12. The method of claim 11, wherein the one or more training parameters include a video of the one or more skills, an instruction associated with the one or more skills, a side-by-side comparison between a practitioner feature of the one or more skills and a target feature of the one or more skills, or a combination thereof.

13. The method of claim 9, wherein the score is an aggregated score based on a plurality of skills.

14. The method of claim 9, further comprising:
receiving historical data for the clinical procedure;
training one or more machine learning systems, using at least the historical data, to identify at least one of the one or more skills; and
training the one or more machine learning systems to generate the quality level and the score.

15. The method of claim 9, wherein the input clinical data further includes at least one of audio data or text data.

16. A processor, comprising:
one or more circuits to:
receive input clinical data for a clinical procedure, the input clinical data including at least a sequence of frames corresponding to video data collected during the clinical procedure;
identify, from one or more segmented regions from at least a portion of the sequence of frames using one or more trained classifiers, one or more features of interest and associated time stamps corresponding to one or more actions for performing one or more skills, wherein the one or more skills are correlated to one or more of an identified object in the one or more segmented regions, an identified tool in the one or more segmented regions, an identified parameter associated with a body part depicted in the one or more segmented regions, or a correlation between the associated time stamps and the body part depicted in the one or more segmented regions;
determine, based on one or more metrics and the associated time stamps for the one or more skills, a quality level for the one or more skills, the quality level corresponding to the one or more actions associated with the one or more skills over a period of time identified by a presence of the one or more features of interest across a plurality of time stamps, and the one or more metrics being selected based on at least one of a correlation between the one or more skills and the clinical outcome, a correlation between the one or more skills and a recommendation, or a performance factor associated with performing the one or more skills; and
provide a score for a practitioner associated with the clinical procedure based, at least in part, on the quality level.

17. The processor of claim 16, wherein the one or more circuits are further to:
determine, from the one or more features of interest, a duration of time associated with executing the one of more skills.

18. The processor of claim 16, wherein the one or more circuits are further to:
receive a training request, the training request including identification of an action coded within the one or more segmented regions associated with a skill of the one or more skills having a respective quality level below a threshold; and
provide, responsive to the training request, one or more training parameters for the action.

19. The processor of claim 18, wherein the one or more training parameters include a video of the one or more skills, an instruction associated with the one or more skills, or a combination thereof.

20. The processor of claim 16, wherein the score is an aggregated score based on a plurality of skills.
